(19) United States
(12) Patent Application Publication
(10) Pub. No.: US 2019/0142891 A1
(43) Pub. Date: May 16, 2019

(54) FERMENTATION PRODUCT OF MUNG BEAN HULL AND USES OF THE SAME

(71) Applicant: TCI CO., LTD., Taipei (TW)

(72) Inventors: Yung-Hsiang LIN, Taipei (TW); Yu-Ting LIN, Taipei (TW)

(21) Appl. No.: 16/182,198

(22) Filed: Nov. 6, 2018

Related U.S. Application Data
(60) Provisional application No. 62/584,418, filed on Nov. 10, 2017.

(30) Foreign Application Priority Data
Oct. 4, 2018 (TW) 107135019

(51) Int. Cl. A61K 36/48; A61P 3/10; A61K 9/00; A61P 9/00; A61P 9/12

(52) U.S. Cl. CPC A61K 36/48; A61P 3/10; A61K 9/0019; A61K 9/0053; A61K 2236/53; A61P 9/12; A61K 2236/19; A61K 2236/15; A61P 9/00

(57) ABSTRACT

A fermentation product of mung bean hull and its uses are provided, wherein the fermentation product is provided by fermenting a mung bean hull and/or an extract of mung bean hull in the present of Lactobacillus plantarum. And, the fermentation product of mung bean hull is used for regulating blood sugar, assisting in regulation of blood pressure, assisting in regulation of blood lipid, preventing hypertension, reducing blood pressure, preventing hypertriglyceridemia, reducing blood lipid, preventing hyperglycemia, reducing blood sugar, preventing cardiovascular disease, treating cardiovascular disease, preventing diabetes, and/or treating diabetes.

### CROSS REFERENCE TO RELATED APPLICATIONS

This application claims priority to U.S. Provisional Application Ser. No. 62/584,418 filed on Nov. 10, 2017, in the United States Patent and Trademark Office, and to Taiwan Patent Application No. 107135019 filed on Oct. 4, 2018, in the Taiwan Intellectual Property Office, the disclosure of which are incorporated herein in its entirety by reference.

### FIELD OF THE INVENTION

The present invention relates to a fermentation product of mung bean hull and uses of the same, wherein the fermentation product of mung bean hull is provided by fermenting a mung bean hull and/or an extract of mung bean hull in the presence of Lactobacillus plantarum, and the fermentation product is used for preventing hypertension, reducing blood pressure, assisting in regulation of blood pressure, preventing hypertriglyceridemia, reducing blood lipid, assisting in regulation of blood lipid, preventing hyperglycemia, reducing blood sugar, regulating blood sugar, preventing cardiovascular disease, treating cardiovascular disease, preventing diabetes and treating diabetes. The fermentation product is also used for increasing the expression of NOS3 gene, decreasing the expression of SREBP-1c gene, decreasing the expression of SCD1 gene, and increasing the plasma membrane content of GLUT4 protein.

### BACKGROUND OF THE INVENTION

Hypertension also known as high blood pressure, is a long-term medical condition in which the blood pressure in the arteries is persistently elevated. In most cases of hypertension patients, no significant symptom would be observed, and thus hypertension is often called “the silent killer.” According to estimates from the World Hypertension League (WHL) in 2010, about 18% of deaths in the world were relevant to hypertension.

Hypertriglyceridemia refers to an excessive level of lipids (e.g., cholesterol, triacylglycerol, etc.) in the blood. When the level of blood lipids is too high, lipids would easily accumulate on the vascular wall, and thus would gradually narrow the blood vessel channel, cause arteriosclerosis, and even result in thrombosis and/or vascular occlusion, thereby leading to the cardiovascular diseases such as angina, myocardial infarction, brain hemorrhage, stroke, etc.

The increment of blood sugar is mainly caused by the nutrition uptake from food. After food enters the gastrointestinal tract, it will be digested and absorbed, and the glucoses thus obtained will go into the blood stream and cause an increased level of blood glucose. Generally, insulin secreted by pancreas can promote the uptake of blood glucose (i.e., blood sugar) into cells, and thus can gradually reduce the blood sugar level to normal. However, an insufficient insulin secretion or insulin resistance may cause the blood sugar level to drop at a too slow rate and may even keep the blood sugar at a level high, that is so-called hyperglycemia or high blood sugar. The high blood sugar condition would develop into diabetes easily if it is not controlled efficiently.

It was revealed by researches that, as compared to normal people, patients suffering from hypertension, hypertriglyceridemia and/or hyperglycemia have a higher risk in chronic diseases (e.g., diabetes and renal disease) and cardiovascular diseases (e.g., myocardial infarction and stroke). It is often for patients to get two or all of hypertension, hypertriglyceridemia and hyperglycemia, such condition causes an even sever threat to the patients' health. Currently, the therapies for patients suffering from hypertension, hypertriglyceridemia and/or hyperglycemia primarily rely on drugs, while drugs can only control but not cure those diseases completely. Hence, most of such patients need to take drug(s) for a long period of time or even the entire lifetime; and patients suffering from hypertension, hypertriglyceridemia and hyperglycemia simultaneously need to take more than one drug to maintain the balances of blood pressure, blood lipid and blood sugar. The situation of taking several drugs would affect the life quality of patients seriously, because each drug has its own characteristics and may cause different side effect(s). Therefore, the industries have devoting in the development of a drug or method for preventing, treating or regulating hypertension, hypertriglyceridemia and/or hyperglycemia conveniently and effectively.

It is known that a decreased expression of NOS3 gene, an overexpression of SREBP-1c gene, an overexpression of SCD1 gene and/or a decreased content of GLUT4 protein on the plasma membrane would lead to the occurrences of hypertension, hypertriglyceridemia, hyperglycemia, cardiovascular disease and/or diabetes. Inventors of the present invention discovered that a fermentation product of mung bean hull, which is provided by fermenting a mung bean hull or an extract of mung bean hull in the presence of Lactobacillus plantarum, is effective in increasing the expression of NOS3 gene, decreasing the expression of SREBP-1c gene, decreasing the expression of SCD1 gene and increasing the plasma membrane content of GLUT4 protein, and can be used for preventing hypertension, reducing blood pressure, assisting in regulation of blood pressure, preventing hypertriglyceridemia, reducing blood lipid, assisting in regulation of blood lipid, preventing hyperglycemia, reducing blood sugar, regulating blood sugar, preventing cardiovascular disease, treating cardiovascular disease, preventing diabetes and/or treating diabetes.

### SUMMARY OF THE INVENTION

An objective of the present invention is to provide a fermentation product of mung bean hull, which is provided by fermenting a mung bean hull and/or an extract of mung bean hull in the presence of Lactobacillus plantarum. Preferably, the fermentation is conducted in the presence of an externally added first solvent. Preferably, the extract of mung bean hull is provided by extracting a mung bean hull with a second solvent. More preferably, the first and second solvent are independently water.

Another objective of the present invention is to provide a use of the aforesaid fermentation product of mung bean hull in the manufacture of a pharmaceutical composition, wherein the pharmaceutical composition is for at least one of preventing hypertension, reducing blood pressure, preventing hypertriglyceridemia, reducing blood lipid, preventing hyperglycemia, reducing blood sugar, preventing cardiovascular disease, treating cardiovascular disease, preventing diabetes, and treating diabetes. Preferably, the pharmaceutical composition is for reducing insulin resistance. Preferably, the cardiovascular disease is at least one of myocardial infraction and left ventricular dysfunction. Preferably, the pharmaceutical composition is provided in a form for oral administration, transdermal administration, or injection administration.

Still another objective of the present invention is to provide a use of the aforesaid fermentation product of mung bean hull in at least one of regulating blood sugar, assisting in regulation of blood pressure and assisting in regulation of blood lipid. Preferably, the fermentation product of mung bean hull is taken through oral route.

Yet another objective of the present invention is to provide a use of the aforesaid fermentation product of mung bean hull in the manufacture of a pharmaceutical composition, wherein the pharmaceutical composition is for at least one of increasing the expression of NOS3 gene, decreasing the expression of SREBP-1c gene, decreasing the expression of SCD1 gene, and increasing the plasma membrane content of GLUT4 protein. Preferably, the pharmaceutical composition is provided in a form for oral administration, transdermal administration, or injection administration.

Yet another objective of the present invention is to provide a pharmaceutical composition for preventing hypertension, reducing blood pressure, preventing hypertriglyceridemia, reducing blood lipid, preventing hyperglycemia, reducing blood sugar, preventing cardiovascular disease, treating cardiovascular disease, preventing diabetes and/or treating diabetes. The pharmaceutical composition comprises an effective amount of the aforesaid fermentation product of mung bean hull. Preferably, the pharmaceutical composition is for reducing insulin resistance. Preferably, the cardiovascular disease is at least one of myocardial infraction and left ventricular dysfunction. Preferably, the pharmaceutical composition is provided in a form for oral administration, transdermal administration, or injection administration.

Yet another objective of the present invention is to provide a food composition for regulating blood sugar, assisting in regulation of blood pressure and/or assisting in regulation of blood lipid. The food composition comprises an effective amount of the aforesaid fermentation product of mung bean hull. Preferably, the food composition is a health food, a daily supplement, a functional food, a nutritional supplement, or a special nutritional food.

Yet another objective of the present invention is to provide a pharmaceutical composition for increasing the expression of NOS3 gene, decreasing the expression of SREBP-1c gene, decreasing the expression of SCD1 gene, and/or increasing the plasma membrane content of GLUT4 protein. The pharmaceutical composition comprises an effective amount of the aforesaid fermentation product of mung bean hull. Preferably, the pharmaceutical composition is provided in a form for oral administration, transdermal administration, or injection administration.

Yet another objective of the present invention is to provide a method for at least one of preventing hypertension, reducing blood pressure, assisting in regulation of blood pressure, preventing hypertriglyceridemia, reducing blood lipid, assisting in regulation of blood lipid, preventing hyperglycemia, reducing blood sugar, regulating blood sugar, preventing cardiovascular disease, treating cardiovascular disease, preventing diabetes, and treating diabetes. The method comprises administering to a subject in need an effective amount of the aforesaid fermentation product of mung bean hull. In the method of the present invention, the fermentation product of mung bean hull can be administered as the pharmaceutical or food composition described above. Preferably, the method is for reducing insulin resistance. Preferably, the cardiovascular disease is at least one of myocardial infraction and left ventricular dysfunction.

Yet another objective of the present invention is to provide a method for increasing the expression of NOS3 gene, decreasing the expression of SREBP-1c gene, decreasing the expression of SCD1 gene, and/or increasing the plasma membrane content of GLUT4 protein. The method comprises administering to a subject in need an effective amount of the aforesaid fermentation product of mung bean hull. In the method of the present invention, the fermentation product of mung bean hull can be administered as the pharmaceutical composition described above.

The detailed technology and preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 shows the influence of the fermentation product of mung bean hull of the present invention on the expression of NOS3 gene, wherein the HUVEC cells in the “control group” were cultured in a medium free of fermentation product of mung bean hull and extract of mung bean hull for 48 hours, and those in the “fermentation product group” and “extract group” were independently cultured in a medium externally added with fermentation product of mung bean hull and extract of mung bean hull, respectively, for 48 hours;

FIG. 2 shows the influence of the fermentation product of mung bean hull of the present invention on the expressions of SREBP-1c and SCD1 genes, wherein the HepG2 cells in the “control group” were cultured in a medium free of fermentation product of mung bean hull and extract of mung bean hull for 48 hours, and those in the “fermentation product group” and “extract group” were independently cultured in a medium externally added with fermentation product of mung bean hull and extract of mung bean hull, respectively, for 48 hours; and

FIG. 3 shows the influence of the fermentation product of mung bean hull of the present invention on the plasma membrane content of GLUT4 protein, wherein the HepG2 cells in the “blank group” were cultured in a medium free of insulin, fermentation product of mung bean hull and extract of mung bean hull for 48 hours, those in the “control group” were cultured in a medium free of insulin, fermentation product of mung bean hull and extract of mung bean hull for 24 hours, and then cultured in a medium externally added with insulin for 24 hours, and those in the “extract group” and “fermentation product group” were independently cultured in a medium externally added with extract of mung bean hull and fermentation product of mung bean hull, respectively, for 24 hours, and then cultured in a medium externally added with insulin for 24 hours.

### DESCRIPTION OF THE PREFERRED EMBODIMENT

The following will describe some of the embodiments of the present invention in detail. However, without departing from the spirit of the present invention, the present invention may be embodied in various embodiments and should not be limited to the embodiments described in the specification or defined in the appended claims.

Unless otherwise indicated herein, the expressions “a,” “an,” “the,” or the like recited in the specification of the present invention (especially in the claims) are intended to include both the singular and plural forms. The term “treat” or “treating” recited in this specification should not be construed as treating a subject until the subject completely recovered, but should include maintaining the progression or symptoms of the diseases in a substantially static state, increasing the recovery rate of a subject, alleviating the severity of a particular condition of illness, or increasing the life quality of patients. The term “prevent” or “preventing” recited in this specification refers to inhibiting or avoiding a particular condition of illness from breaking out, or maintaining good health in a sensitive subject to tolerate diseases. The term “regulate”, “regulating”, or “regulation” recited in this specification refers to upregulating (includes inducing, stimulating, and enhancing) or downregulating (includes inhibiting and weakening) the physiological functions in a subject toward a normal state. The term “subject” recited in this specification refers to a mammalian, including human and non-human animals (e.g., dogs or cats).

As described above, it is known that a decreased expression of NOS3 gene is relevant to the occurrences of hypertension and cardiovascular diseases (e.g., myocardial infarction and left ventricular dysfunction). Therefore, if the expression of NOS3 gene can be increased effectively, the following effects could be provided: preventing hypertension, reducing blood pressure, assisting in regulation of blood pressure, treating or preventing cardiovascular diseases (including myocardial infarction and left ventricular dysfunction). These can be noted in “NOS3 Genotype-Dependent Correlation Between Blood Pressure and Physical Activity. Hypertension. 41: 355-360 (2003)” and “Depletion of circulating blood NOS3 increases severity of myocardial infarction and left ventricular dysfunction. Basic Res Cardiol. 109(1):398 (2014)”, which are entirely incorporated hereinto by reference.

It is known that an increased expression of SREBP-1c gene may result in an enhancement of the fatty acid synthesis, and an overexpression of SREBP-1c gene may lead to hypertriglyceridemia. Therefore, if the expression of SREBP-1c gene can be inhibited effectively, the following effects could be provided: preventing hypertriglyceridemia, reducing blood lipid, and assisting in regulation of blood lipid. These can be noted in “ADD1/SREBP-1c Is Required in the Activation of Hepatic Lipogenic Gene Expression by Glucose. MOLECULAR AND CELLULAR BIOLOGY. 19(5): 3760-3768 (1999)”, which is entirely incorporated hereinto by reference.

It was revealed by researches that the deficiency of SCD1 gene can prevent the occurrence of hypertriglyceridemia, reduce hepatic triglyceride accumulation, and increase plasma high-density lipoprotein cholesterol. Therefore, if the expression of SCD1 gene can be inhibited effectively, the following effects could be provided: preventing hypertriglyceridemia, reducing blood lipid, and assisting in regulation of blood lipid. These can be noted in “Stearoyl-Coenzyme A Desaturase 1 Deficiency Protects against Hypertriglyceridemia and Increases Plasma High-Density Lipoprotein Cholesterol Induced by Liver X Receptor Activation. American Society for Microbiology. 26(18): 6786-6798 (2006)”, which is entirely incorporated hereinto by reference.

GLUT4 protein is a glucose transporter. When GLUT4 protein translocate from cytoplasm to cell membrane, the glucose uptake of cell will be enhanced. Therefore, an increment in the plasma membrane content of GLUT4 protein is beneficial to the glucose uptake of cells and the regulation of blood sugar, and thus can provide the following effects: preventing hyperglycemia, reducing blood sugar, regulating blood sugar, treating or preventing diabetes (especially type 2 diabetes). These can be noted in “REGULATED TRANSPORT OF THE GLUCOSE TRANSPORTER GLUT4. MOLECULAR CELL BIOLOGY. 3: 267-277 (2002)” and “GLUT4 Is Reduced in Slow Muscle Fibers of Type 2 Diabetic Patients. Diabetes. 50: 1324-1329 (2001)”, which are entirely incorporated hereinto by reference.

Inventors of the present invention incidentally discovered that the fermentation product of mung bean hull, which is provided by fermenting a mung bean hull and/or an extract of mung bean hull in the presence of Lactobacillus plantarum, can effectively increase the expression of NOS3 gene, decrease the expression of SREBP-1c gene, decrease the expression of SCD1 gene and increase the plasma membrane content of GLUT4 protein. Therefore, the present invention relates to a provision of a novel fermentation product of mung bean hull and the uses of the same. The uses include: using the fermentation product of mung bean hull in regulating blood sugar, assisting in regulation of blood pressure, assisting in blood lipid, using the fermentation product of mung bean hull in manufacturing a pharmaceutical composition, providing a food composition or a pharmaceutical composition containing an effective amount of the fermentation product of mung bean hull, and providing a method of administering to a subject in need an effective amount of the aforesaid food composition or pharmaceutical composition. The fermentation product of mung bean hull in accordance with the present invention is provided by fermenting a mung bean hull and/or an extract of mung bean hull in the presence of Lactobacillus plantarum. The food composition provided in accordance with the present invention is for regulating blood sugar, assisting in regulation of blood pressure, and/or assisting in regulation of blood lipid. The pharmaceutical composition provided in accordance with the present invention is for at least one of preventing hypertension, reducing blood pressure, preventing hypertriglyceridemia, reducing blood lipid, preventing hyperglycemia, reducing blood sugar, preventing cardiovascular disease, treating cardiovascular disease, preventing diabetes (especially type 2 diabetes), and treating diabetes (especially type 2 diabetes). Furthermore, the pharmaceutical composition and method provided in accordance with the present invention are also for increasing the expression of NOS3 gene, decreasing the expression of SREBP-1c gene, decreasing the expression of SCD1 gene, and/or increasing the plasma membrane content of GLUT4 protein.

The fermentation product of mung bean hull in accordance with the present invention is provided by fermenting a mung bean hull and/or an extract of mung bean hull in the presence of Lactobacillus plantarum. For example, the fermentation product of mung bean hull can be provided by, but is not limited to: (i) dispersing mung bean hulls into a solvent evenly to provide a mixture, and inoculating Lactobacillus plantarum into the mixture and fermenting the inoculated mixture; or (ii) extracting mung bean hulls with a solvent to provide an extract, and inoculating Lactobacillus plantarum into the extract and fermenting the inoculated extract. In some embodiments of the present invention, the solvent is a polar solvent selected from a group consisting of water, alcohols (e.g., C1-C4 alcohols), and combinations thereof. Preferably, the solvent is water, ethanol, or a combination thereof. Generally, there is no limitation of the amount of the solvent as long as the mung bean hull can be evenly dispersed in the solvent. For example, the extraction could be conducted with the use of the solvent and the mung bean hulls at a weight ratio of 20:1 (solvent:mung bean hulls).

Optionally, one or more operations such as heating, cooling, stirring, and ultrasonication could be performed before or during the extracting or mixing step. For example, the extracting or mixing step could be conducted at an elevated temperature ranging from 75° C. to 95° C. In one embodiment of the present invention, the extraction was conducted with the use of water as a solvent and the weight ratio of water:mung bean hulls is 20:1, and then the mixture thus obtained was maintained at 85° C. for 60 minutes to provide a crude extract, and an extract of mung bean hull was provided by subjecting the crude extract to a solid-liquid separation. Both the crude extract and the extract of mung bean hull could be used for the following fermentation.

In the fermentation indicated above, there is no particular limitation of the inoculating amount of Lactobacillus plantarum. For example, if the fermentation is conducted by using the extract of mung bean hull as the fermentation material, the amount of Lactobacillus plantarum could be that the weight ratio of fermentation material:bacterial powder of Lactobacillus plantarum is 1000:1. Furthermore, in some embodiments of the present invention, an extract of mung bean hull was fermented in the presence of Lactobacillus plantarum at a temperature ranging from 25° C. to 35° C. for 48 to 72 hours.

Optionally, to enhance the convenience of using the extract or fermentation product, the operations such as solid-liquid separation (e.g., filtration and centrifugation), vacuum concentration, drying (e.g., hot-air drying, freeze-drying and spray-drying) and sterilization could be conducted after the aforesaid extraction or fermentation completed.

The pharmaceutical composition provided in accordance with the present invention could be administered to a subject in need systemically or topically, and could be delivered by various drug delivery systems (DDSs), such as oral drug delivery system, transdermal drug delivery system, etc. For example, to enhance bioavailability, control drug release speed, target the lesion precisely and reduce side effects, the pharmaceutical composition could be delivered by a liposome, a microcapsule, nanoparticles, microneedles, but is not limited thereby.

Depending on the desired purpose(s), the pharmaceutical composition of the present invention could be provided in any suitable form without particular limitations. For example, the pharmaceutical composition could be provided in a form suitable for administering to a subject in need by oral administration, transdermal administration or injection administration (including intravenous injection, intramuscular injection, subcutaneous injection, etc.), but is not limited thereby. Depending on the form and purpose(s), a suitable carrier could be chosen and used to provide the pharmaceutical composition or a food product composition. Examples of the carrier include excipients, diluents, auxiliaries, stabilizers, absorbent retarders, disintegrating agents, hydrotropic agents, emulsifiers, antioxidants, adhesives, binders, tackifiers, dispersants, suspending agents, lubricants, hygroscopic agents, etc.

As a form for oral administration, the pharmaceutical composition of the present invention could comprise any pharmaceutically acceptable carrier that will not adversely affect the desired effects of the active ingredient (i.e., fermentation product of mung bean hull). Examples of suitable carrier include, but are not limited to, water, saline, dextrose, glycerol, ethanol or its analogs, oil (such as olive oil, castor oil, cottonseed oil, peanut oil, corn oil, germ oil), starch, polyethylene glycol, kaolinite, bentonite, sodium citrate, gelatin, agar, carboxymethyl cellulose, gum arabic, algin and its salts, glyceryl monostearate, calcium stearate, and combinations thereof. The pharmaceutical composition could be provided by any suitable method in any suitable form for oral administration, such as in the form of a tablet (e.g., sugar-coated tablet), a pill, a capsule, granules, a pulvis, a fluid extract, a solution, a syrup, a suspension, a tincture, etc.

As a form for transdermal administration, the pharmaceutical composition of the present invention could also comprise any pharmaceutically acceptable carrier that will not adversely affect the desired effects of the active ingredient (i.e., fermentation product of mung bean hull). Examples of suitable carrier include, but are not limited to, water, mineral oil, and hyaluronic acid. The pharmaceutical composition could be provided by any suitable method in any suitable form for transdermal administration, such as in a form of a patch (e.g., a microneedle patch), but is not limited thereby.

As a form of injection or drip suitable for injection administration, the pharmaceutical composition provided in accordance with the present invention could comprise one or more ingredient(s), such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition as an intravenous infusion, an emulsified intravenous infusion, a powder for injection, a suspension for injection, or a powder suspension for injection, etc. Alternatively, the pharmaceutical composition could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to a subject in need.

Depending on the needs, age, body weight, and health conditions of the subject, the pharmaceutical composition provided in accordance with the present invention could be administered at various frequencies, such as once a day, multiple times a day, or once every few days, etc. The concentration of fermentation product of mung bean hull in the pharmaceutical composition could be adjusted depending on the requirements of practical application. In addition, the pharmaceutical composition could optionally further comprise one or more other active ingredient(s) (e.g., antihypertensive agents, hypolipidemic agents, hypoglycemic agents, etc.), or to be used in combination with a medicament comprising one or more other active ingredient(s), to further enhance the effects of the pharmaceutical composition, or to increase the application flexibility and application adaptability of preparation thus provided, as long as the other active ingredients do not adversely affect the desired effects of the active ingredients of the present invention (i.e., fermentation product of mung bean hull).

Optionally, the pharmaceutical composition or food composition provided in accordance with the present invention could further comprise a suitable amount of additives, such as a flavoring agent, a toner, or a coloring agent for enhancing the palatability and the visual perception of the pharmaceutical composition or food composition, and/or a buffer, a conservative, a preservative, an antibacterial agent, or an antifungal agent for improving the stability and storability of the pharmaceutical composition or food composition.

The food combination provided in accordance with the present invention could be a health food, a dietary supplement, a functional food, a nutritional supplement food or a special nutritional food, and can be manufactured as dairy products, meat products, breadstuff, pasta, cookies, troche, capsules, fruit juices, teas, sports beverages, nutritional beverages, etc., but is not limited thereby. Preferably, the food composition provided in accordance with the present invention is a health food.

Depending on the age, body weight, and health conditions of the subject, the food composition provided in accordance with the present invention could be taken at various frequencies, such as once a day, multiple times a day, or once every few days, etc. The amount of fermentation product of mung bean hull in the food composition provided in accordance with the present invention could also be adjusted, preferably to the amount that it should be taken daily, depending on the specific population.

The recommended daily dosage, use standards and use conditions for a specific population (e.g., patients with hypertension, hypertriglyceridemia, hyperglycemia, heart diseases, renal diseases or stroke, and pregnant woman), or the recommendations for a use in combination with another food product or medicament could be labeled on the exterior package of health food, dietary supplement, functional food, nutritional supplement food and/or special nutritional food provided in accordance with the present invention. Thus, it is suitable for the users to take the health food, dietary supplement, functional food, nutritional supplement food and/or special nutritional food by him- or herself safely and securely without the instructions of a doctor, pharmacist or related executive.

In the use of fermentation product of mung bean hull in regulating blood sugar, assisting in regulation of blood pressure, and/or assisting in regulation of blood lipid in accordance with the present invention, the fermentation product of mung bean hull could be provided as a food product composition, and the administration type, administration route, administration form, administration frequency and uses of the food product composition are all in line with the above descriptions.

As described above, the present invention also provides a method for at least one of preventing hypertension, reducing blood pressure, assisting in regulation of blood pressure, preventing hypertriglyceridemia, reducing blood lipid, assisting in regulation of blood lipid, preventing hyperglycemia, reducing blood sugar, regulating blood sugar, preventing cardiovascular disease, treating cardiovascular disease, preventing diabetes, and treating diabetes, comprising administering to a subject in need an effective amount of the fermentation product of mung bean hull, wherein the term “a subject in need” refers to a subject suffering from dysarteriotony, dyslipidemia, abnormal level of blood sugar, cardiovascular disease, and/or diabetes, or a subject with high risk in the above diseases. In this method, the fermentation product of mung bean hull could be administered to the subject in need as a pharmaceutical composition or a food product composition ad described above, and the administration type, administration route, administration form, administration frequency and uses of the pharmaceutical composition and the food product composition are also all in line with the above descriptions.

The present invention also provides a method for at least one of increasing the expression of NOS3 gene, decreasing the expression of SREBP-1c gene, decreasing the expression of SCD1 gene, and increasing the plasma membrane content of GLUT4 protein, comprising administering to a subject in need an effective amount of the fermentation product of mung bean hull, wherein “a subject in need” refers to a subject whose NOS3 gene is deleted, mutated or expressed at low level, SREBP-1c gene is over-expressed, SCD1 gene is over-expressed, and/or GLUT4 protein is expressed at low level on the plasma membrane. In this method, the fermentation product of mung bean hull could be administered to the subject in need as a pharmaceutical composition described above, and the administration type, administration route, administration form, administration frequency and uses of the pharmaceutical composition are also all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Preparation Example

A. Preparation of Extract of Mung Bean Hull

Mung beans (purchased from Chaio Lien Foods Processing Factory Ltd., Taiwan) were shelled by a shelling machine at a temperature of no more than 90° C. Then, the mung bean hulls thus obtained were crushed and subjected to the following steps to provide an extract of mung bean hull:
1. Mixing the mung bean hulls with water at a weight ratio of 1:20 (mung bean hulls:water) evenly, and maintaining the mixture thus obtained at 85° C. for 60 minutes to provide a crude extract;
2. Filtering the crude extract to provide a filtrate (i.e., the extract of mung bean hull for use in [Preparation Example B]); and
3. Freeze-drying filtrate to provide a dry matter (i.e., the extract of mung bean hull for use in the Examples 1 to 3).

B. Preparation of Fermentation Product of Mung Bean Hull

The extract of mung bean hull provided by [Preparation Example A] was subjected to the following steps to provide a fermentation product of mung bean hull:
1. Inoculating Lactobacillus plantarum (deposited at German Collection of Microorganisms and Cell Cultures (Deutsche Sammlung von Mikroorganismen and Zellkulturen, DSMZ) under the accession number DSM 32451 on Mar. 13, 2017) into the extract of mung bean hull provided by [Preparation Example A] at a weight ratio of 1:1000 (Lactobacillus plantarum:extract of mung bean hull), and maintaining the inoculated extract at a temperature ranging from 25° C. to 35° C. for 48 hours to provide a fermentation liquid;
2. Filtering the fermentation liquid to provide a filtrate; and
3. Freeze-drying the filtrate to provide a dry matter (i.e., the fermentation product of mung bean hull for use in Examples 1 to 3).

C. Preparation of HUVEC Cell Medium

The Medium 200 (purchased from Gibco, product no.: M-200-500) was mixed with low serum growth supplement (LSGS; purchased from Gibco) at a volume ratio of 9:1 (Medium 200:LSGS) evenly to provide a HUVEC cell medium for use in the following examples.

D. Preparation of HepG2 Cell Medium

The Dulbecco's Modified Eagle's Medium (DMEM; purchased from Gibco) was mixed with fetal bovine serum (FBS; purchased from Gibco) a volume ratio of 9:1 (DMEM:FBS) evenly to provide a HepG2 cell medium for use in the following examples.

### Example 1

### Effects of Fermentation Product of Mung Bean Hull on Increasing Expression of NOS3 Gene

Human umbilical vein endothelial cells (HUVEC; purchased from BCRC, product no.: H-UV001) were divided into three groups and cultured with the following media at 37° C. for 48 hours:
1. Control group: the HUVEC cell medium provided by [Preparation Example C];
2. Extract group: same as that of the control group, but was externally added with 4 mg of the extract of mung bean hull provided by [Preparation Example A] per mL of the medium; and
3. Fermentation product group: same as that of the control group, but was externally added with 4 mg of the fermentation product of mung bean hull provided by [Preparation Example B] per mL of the medium.

The cells of each group were respectively harvested and subjected to an RNA extraction with an RNA extraction kit (purchased from Geneaid). The RNA of each group was reverse transcribed into cDNA with a SuperScript® III Reverse Transcriptase kit (purchased from Invitrogen). Then, the cDNA of each group was subjected to qPCR (quantitative polymerase chain reaction) by using a StepOne Plus™ System (purchased from ABI) and a KAPA SYBR FAST qPCR Kit (2×) (purchased from KAPA Biosystems) to determine the expression level of NOS3 gene in the cells of each group.

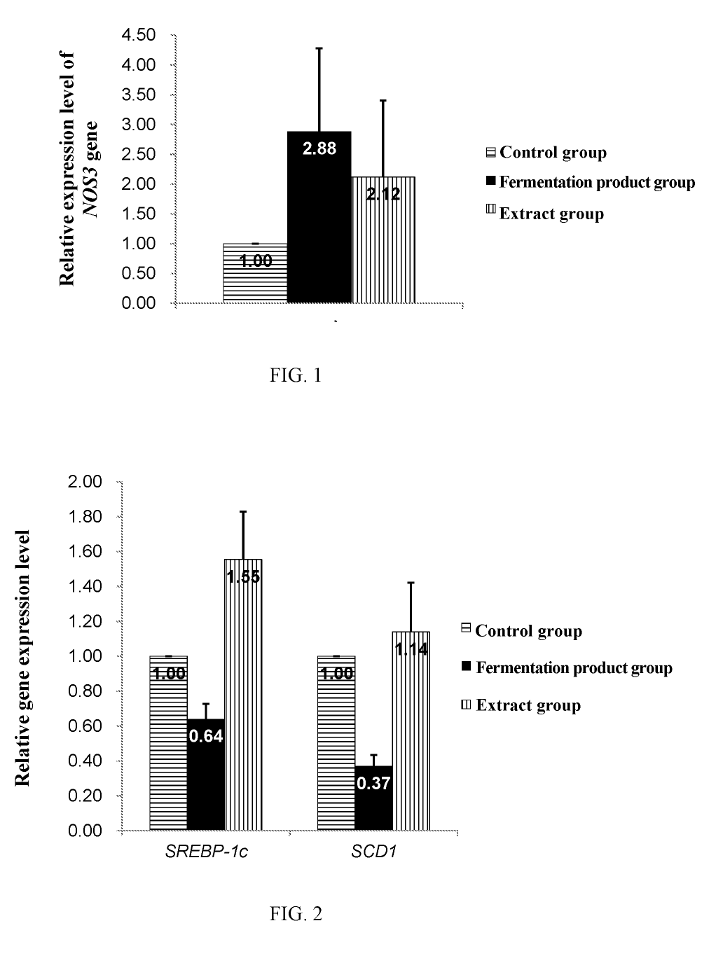

The above experimentation was repeated thrice, and the results of the three experimentations were averaged. Then, the result of control group was used as a basis (i.e., the expression level of NOS3 gene of the control group was set as 1-fold) to calculate the relative gene expression level of the extract group. The results are shown in FIG. 1.

As shown in FIG. 1, the expression level of NOS3 gene in the cells of the extract group and fermentation product group were significantly higher than that of the control group. Furthermore, the expression level of NOS3 gene in the cells of the fermentation product group was higher than that of the extract group. These results indicate that the fermentation product of mung bean hull provided in accordance with the present invention can effectively increase the expression of NOS3 gene, and is more effective than the unfermented extract of mung bean hull. Hence, the fermentation product of mung bean hull provided in accordance with the present invention can be used for preventing hypertension, reducing blood pressure, assisting in regulation of blood pressure, treating or preventing cardiovascular diseases (including myocardial infarction and left ventricular dysfunction).

### Example 2

### Effects of Fermentation Product of Mung Bean Hull on Decreasing Expression of SREBP-1c Gene and SCD1 Gene

HepG2 cells (a human hepatocellular carcinoma cell line; purchased from ATCC; product no. HB-8065™) were divided into three groups and cultured with the following media at 37° C. for 48 hours:
1. Control group: the HepG2 cell medium provided by [Preparation Example D];
2. Extract group: same as that of the control group, but was externally added with 4 mg of the extract of mung bean hull provided by [Preparation Example A] per mL of the medium; and
3. Fermentation product group: same as that of the control group, but was externally added with 4 mg of the fermentation product of mung bean hull provided by [Preparation Example B] per mL of the medium.

The cells of each group were respectively harvested and subjected to the analysis same as that of Example 1 to determine the expression level of SREBP-1c gene and SCD1 gene in the cells of each group. The results are shown in FIG. 2.

As shown in FIG. 2, the expression level of SREBP-1c gene and SCD1 gene in the cells of the extract group were significantly higher than that of the control group. However, the expression level of SREBP-1c gene and SCD1 gene in the cells of the fermentation product group were significantly lower than that of the control group. The results indicate that the fermentation product of mung bean hull provided in accordance with the present invention can effectively decrease the expression of SREBP-1c gene and SCD1 gene. Hence, the fermentation product of mung bean hull provided in accordance with the present invention can be used for preventing hypertriglyceridemia, reducing blood lipid, and assisting in regulation of blood lipid.

### Example 3

### Effects of Fermentation Product of Mung Bean Hull on Increasing Plasma Membrane Content of GLUT4 Protein

2 mL of the HepG2 cell medium provided by [Preparation Example D] was added into each well of a 6-well plate, and then, HepG2 cells were seeded (1×10⁵ cells/well) into each well. The plate was placed under 37° C. for 24 hours. Then, the cell medium in each well was replaced with a DMEM containing 1 μM insulin (purchased from Sigma, product no.: I9278-5ML) and the plate was continuously placed at 37° C. for 72 hours to induce insulin resistance in cells. Thereafter, the cells were divided into four groups and respectively subjected to the following treatments:
1. Blank group: the medium was replaced with a fresh HepG2 cell medium and the cells were continuously cultured at 37° C. for 48 hours;
2. Control group: the medium was replaced with a fresh HepG2 cell medium and the cells were continuously cultured at 37° C. for 24 hours, and then the medium was externally added with insulin (to the final concentration of 0.5 μM) and the cells were continuously cultured at 37° C. for 24 hours;
3. Extract group: the medium was replaced with a HepG2 cell medium externally added with 4 mg of the extract of mung bean hull provided by [Preparation Example A] per mL of the medium and the cells were continuously cultured at 37° C. for 24 hours, and then the medium was replaced with a HepG2 cell medium externally added with insulin (to the final concentration of 0.5 μM) and the cells were continuously cultured at 37° C. for 24 hours; and
4. Fermentation product group: the medium was replaced with a HepG2 cell medium externally added with 4 mg of the fermentation product of mung bean hull provided by [Preparation Example B] per mL of the medium and the cells were continuously cultured at 37° C. for 24 hours, and then the medium was replaced with a HepG2 cell medium externally added with insulin (to the final concentration of 0.5 μM) and the cells were continuously cultured at 37° C. for 24 hours.

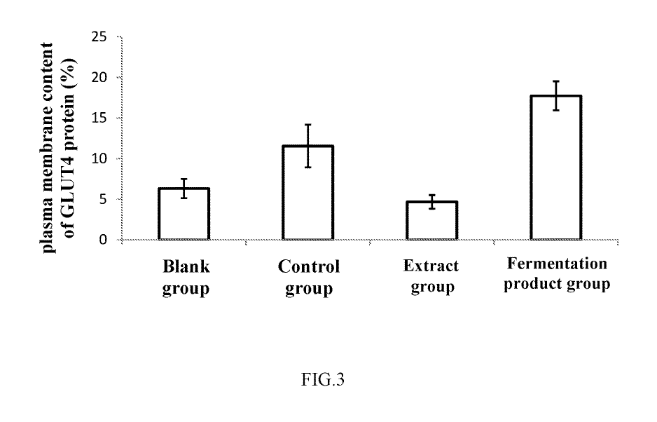

The cells of each group were respectively harvested and subjected to an analysis by using a GLUT4 antibody (purchased from Invitrogen, product no.: MA5-17176) and a flow cytometry (purchased from BD, product no.: Accuri™ C6 Plus) to determine the plasma membrane content of GLUT4 protein in the cells of each group. The above experimentation was repeated thrice, and the results of the three experimentations were averaged. The results are shown in FIG. 3.

As shown in FIG. 3, the plasma membrane content of GLUT4 protein in the cells of the extract group were not significantly different from that of the blank group. However, the plasma membrane contents of GLUT4 protein in the cells of the control group and fermentation product group were significantly higher than that of the blank group. Furthermore, the plasma membrane content of GLUT4 protein in the cells of the fermentation product group was significantly higher than that of the control group. The results indicate that the fermentation product of mung bean hull provided in accordance with the present invention can effectively promote the translocation of GLUT4 protein from cytoplasm to cell membrane and enhance the insulin sensitivity of HepG2 cells, and thus, is effective in promoting blood glucose uptake of cells. Hence, the fermentation product of mung bean hull provided in accordance with the present invention can be used for preventing hyperglycemia, reducing blood sugar, regulating blood sugar, preventing diabetes, and treating diabetes.

What is claimed is:

1. A fermentation product of mung bean hull, which is provided by fermenting a mung bean hull and/or an extract of mung bean hull in the presence of Lactobacillus plantarum.

2. The fermentation product of mung bean hull as claimed in claim 1, wherein the fermentation is conducted in the presence of an externally added first solvent.

3. The fermentation product of mung bean hull as claimed in claim 1, wherein the extract of mung bean hull is provided by extracting a mung bean hull with a second solvent.

4. The fermentation product of mung bean hull as claimed in claim 2, wherein the first solvent is water.

5. The fermentation product of mung bean hull as claimed in claim 3, wherein the second solvent is water.

6. A method for at least one of preventing hypertension, reducing blood pressure, assisting in regulation of blood pressure, preventing hypertriglyceridemia, reducing blood lipid, assisting in regulation of blood lipid, preventing hyperglycemia, reducing blood sugar, regulating blood sugar, preventing cardiovascular disease, treating cardiovascular disease, preventing diabetes and treating diabetes, comprising administering to a subject in need an effective amount of the fermentation product of mung bean hull as claimed in claim 1.

7. The method as claimed in claim 6, which is for at least one of preventing hyperglycemia, reducing blood sugar and regulating blood sugar.

8. The method as claimed in claim 6, which is for at least one of preventing diabetes and treating diabetes.

9. The method as claimed in claim 6, which is for reducing insulin resistance.

10. The method as claimed in claim 6, wherein the fermentation product of mung bean hull is administered to the subject by at least one of oral administration, transdermal administration and injection administration.

11. The method as claimed in claim 7, wherein the fermentation product of mung bean hull is administered to the subject by at least one of oral administration, transdermal administration and injection administration.

12. The method as claimed in claim 8, wherein the fermentation product of mung bean hull is administered to the subject by at least one of oral administration, transdermal administration and injection administration.

13. The method as claimed in claim 9, wherein the fermentation product of mung bean hull is administered to the subject by at least one of oral administration, transdermal administration and injection administration.

14. A method for at least one of increasing the expression of NOS3 gene, decreasing the expression of SREBP-1c gene, decreasing the expression of SCD1 gene, and increasing the plasma membrane content of GLUT4 protein, comprising administering to a subject in need an effective amount of the fermentation product of mung bean hull as claimed in claim 1.

15. The method as claimed in claim 14, wherein the fermentation product of mung bean hull is administered to the subject by at least one of oral administration, transdermal administration and injection administration.
